(19) United States
(12) Patent Application Publication
(10) Pub. No.: US 2012/0330092 A1
(43) Pub. Date: Dec. 27, 2012

(54) DEVICE, SYSTEM, AND METHOD FOR MODULATING CARDIAC FUNCTION

(76) Inventors: Akira Shiose, Pittsburgh, PA (US); Kiyotaka Fukamachi, Mayfield Heights, OH (US); Alex Massiello, Chesterland, OH (US)

(21) Appl. No.: 13/525,815

(22) Filed: Jun. 18, 2012

Related U.S. Application Data
(60) Provisional application No. 61/500,151, filed on Jun. 23, 2011.

(51) Int. Cl. A61N 1/362; A61M 1/10

(52) U.S. Cl. 600/18

(57) ABSTRACT

A therapy delivery device for modulating cardiac function in a subject includes a counterpulsation component, an endovascular lead, and at least one electrode that is physically coupled with the endovascular lead. Each of the counterpulsation component and the endovascular lead includes a proximal end and a distal end. The proximal end of the endovascular lead is physically connected to the distal end of the counterpulsation component.

### RELATED APPLICATION

This application claims the benefit of U.S. Provisional Application Ser. No. 61/500,151, filed Jun. 23, 2011, the entirety of which is hereby incorporated by reference.

### TECHNICAL FIELD

The present invention relates generally to devices and methods for modulating cardiac function, and more particularly to combined counterpulsation and neuromodulatory devices and methods that provide counterpulsation therapy with little or no need for inotropic pharmacologic support.

### BACKGROUND OF THE INVENTION

Diseases causing or resulting in acute heart failure are widespread. The goals of therapy in acute heart failure are often to correct the hemodynamic instability and address decompensation to decrease patient mortality. Current treatments for acute heart failure include pharmacologic treatments, as well as surgical and interventional treatments.

Pharmacologic treatments for acute heart failure include diuretics to reduce ventricular filing pressures (preload), vasodilators to reduce preload and systemic vascular resistance (afterload) and thus ventricular workload, and inotropes to improve cardiac hemodynamics via an increase in contractility and heart rate. Because the increased heart rate and myocardial contractility induced by use of ionotropes also increases myocardial oxygen demand, which aggravates ischemia and precipitates arrhythmias, they are typically reserved for acute heart failure patients with very low cardiac output. In fact, recent guidelines (from 2005 and 2009) jointly issued by the American College of Cardiology and American Heart Association for the management of chronic heart failure state that the use of parenteral and intermittent infusion of positive inotropes is not recommended for long-term treatment of heart failure, even in its advanced stages, and suggest that inotrope use be reserved for palliative care of end-stage heart failure patients in whom the significantly increased mortality risks are traded for a better quality of life (Jessup M. et al., Circulation 119:1977-2016, 2009; and Hunt SA et al., Circulation 112:e154-235, 2005).

Surgical and interventional treatments to increase ventricular function, such as coronary arterial bypass and stenting, valve repair/replacement, left ventricular volume reduction surgery, and mechanical circulatory support device implantation are more invasive approaches reserved for patients not response to pharmacologic treatment.

Pharmacologic and surgical/interventional treatment modalities are often bridged by intra-aortic balloon pumping (IABP) in the face of very low cardiac output and poor coronary perfusion. The IABP is a mechanical device consisting of an intra-aortic balloon placed through a peripheral artery in the aorta. The IABP increases coronary blood flow primarily during ventricular diastole (diastolic augmentation) by inflating the balloon during ventricular diastole and decreases myocardial oxygen demand (systolic unloading) by deflating the balloon during systole. This requires that the IABP be synchronized with the cardiac cycle by timing balloon inflation/deflation with an ECG signal. Although IABP therapy can be effective in decreasing myocardial oxygen demand and increasing myocardial oxygen supply, the concomitant need for inotropic drugs in acute heart failure patients decreases the efficiency of IABP support by further increasing the heart rate of patients which are typically tachycardic. This decreases diastolic times for augmentation of coronary blood flow and decreases the efficiency of synchronization of the ECG signal with balloon inflation/deflation.

### SUMMARY OF THE INVENTION

According to one aspect of the present invention, a therapy delivery device for modulating cardiac function in a subject comprises a counterpulsation component, an endovascular lead, and at least one electrode that is physically coupled with the endovascular lead. Each of the counterpulsation component and the endovascular lead includes a proximal end and a distal end. The proximal end of the endovascular lead is physically connected to the distal end of the counterpulsation component.

According to another aspect of the present invention, a system for modulating cardiac function in a subject comprises a therapy delivery device and a second lead. The therapy delivery device comprises a counterpulsation component, a first endovascular lead, and a first electrode that is physically coupled with the first endovascular lead and has a first polarity. Each of the counterpulsation component and the first endovascular lead includes a proximal end and a distal end. The proximal end of the first endovascular lead is physically connected to the distal end of the counterpulsation component. The second lead has a second electrode physically coupled thereto and a second polarity that is different than the first polarity.

According to another aspect of the present invention, a method for modulating cardiac function in a subject is provided. One step of the method can include providing a therapy delivery device comprising a counterpulsation component, an endovascular lead, and at least one electrode that is physically coupled with the endovascular lead. Each of the counterpulsation component and the endovascular lead includes a proximal end and a distal end. The proximal end of the endovascular lead is physically connected to the distal end of the counterpulsation component. The therapy delivery device is positioned in the vasculature of the subject so that the at least one electrode is in electrical communication with a mixed autonomic nerve target and the counterpulsation component is located in a portion of the descending aorta. The therapy device is then activated, which increases myocardial contractility without increasing the heart rate of the subject.

According to another aspect of the present invention, a method for modulating cardiac function in a subject is provided. One step of the method includes providing a system comprising a therapy delivery device and a second endovascular lead. The therapy delivery device comprises a counterpulsation component, a first endovascular lead, and a first electrode that is physically coupled with the first endovascular lead and has a first polarity. Each of the counterpulsation component and the first endovascular lead includes a proximal end and a distal end. The proximal end of the first endovascular lead is physically connected to the distal end of the counterpulsation component. The second endovascular lead has a second electrode physically coupled thereto and a second polarity that is different than the first polarity. The therapy delivery device is positioned in the vasculature of the subject so that the first electrode is in electrical communication with a mixed autonomic nerve target and the counterpulsation component is located in a portion of the descending aorta. Next, the second endovascular lead is positioned in a blood vessel so that the second electrode is adjacent the first electrode. The system is then activated so that myocardial contractility is increased without an increase in the heart rate of the subject.

According to another aspect of the present invention, a method for modulating cardiac function in a subject is provided. One step of the method includes providing a system comprising a therapy delivery device and an epivascular lead. The therapy delivery device comprises a counterpulsation component, an endovascular lead, and a first electrode that is physically coupled with the endovascular lead and has a first polarity. Each of the counterpulsation component and the endovascular lead includes a proximal end and a distal end. The proximal end of the endovascular lead is physically connected to the distal end of the counterpulsation component. The epivascular lead has a second electrode physically coupled thereto and a second polarity that is different than the first polarity. The therapy delivery device is positioned in the vasculature of the subject so that the first electrode is in electrical communication with a mixed autonomic nerve target and the counterpulsation component is located in a portion of the descending aorta. Next, the epivascular lead is percutaneously positioned so that the second electrode is adjacent the first electrode. The system is then activated so that myocardial contractility is increased without an increase in the heart rate of the subject.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 is a schematic illustration of a human heart showing cardiac autonomic innervations;

FIG. 2 is an alternative schematic illustration showing cardiac autonomic innervations of a human heart;

FIG. 3 is another alternative schematic illustration showing cardiac autonomic innervations of a human heart;

FIG. 4 is a perspective view of a therapy delivery device constructed in accordance with one aspect of the present invention;

FIGS. 5A-B show an intra-aortic balloon pump in an inflated configuration (FIG. 5A) during diastole and a deflated configuration (FIG. 5B) during systole;

FIG. 6 is a magnified perspective view of the endovascular lead component of the therapy delivery device (FIG. 4) comprising multiple electrodes;

FIG. 7 is a schematic illustration showing operation of the therapy delivery device (FIG. 4) in the vasculature of a subject during systole;

FIG. 8 is a schematic illustration showing operation of a therapy delivery device (FIG. 4) in systole constructed in accordance with another aspect of the present invention that includes a second endovascular lead positioned in the vasculature; and

FIG. 9 is a schematic illustration showing operation of a therapy delivery device (FIG. 4) in systole constructed in accordance with another aspect of the present invention that includes an epivascular lead.

### DETAILED DESCRIPTION

Unless otherwise defined, all technical terms used herein have the same meaning as commonly understood by one of ordinary skill in the art to which the present invention pertains.

In the context of the present invention, the term “mixed autonomic nerve target” can refer to any tissue or tissues of the autonomic nervous system (ANS) that comprise(s) at least one nerve fiber of the sympathetic nervous system (SNS) and at least one nerve fiber of the parasympathetic nervous system (PNS).

As used herein, the term “heart failure” can refer to acute or chronic heart failure, and can include NYHA Class II, III or IV.

As used herein, the terms “modulate” or “modulating” when used in the context of neuromodulation can refer to causing a change in neuronal activity, chemistry, and/or metabolism. The change can refer to an increase, decrease, or even a change in a pattern of neuronal activity. The terms may refer to either excitatory or inhibitory stimulation, or a combination thereof, and may be at least electrical, magnetic, optical or chemical, or a combination of two or more of these. The terms “modulate” or “modulating” can also be used to refer to a masking, altering, overriding, or restoring of neuronal activity.

As used herein, the term “electrical communication” can refer to the ability of an electric field generated by an electrode or electrode array to be transferred, or to have a neuromodulatory effect, within and/or on at least one nerve, neuron, and/or nervous tissue of the ANS.

As used herein, the term “intravascular target site” can refer to a desired anatomical location at which a therapy delivery device may be positioned. The intravascular target site can comprise a variety of anatomical locations innervated by, or in electrical communication with, nervous tissue of the ANS. For example, the intravascular target site can comprise an intra-arterial location in electrical communication with a mixed autonomic nerve target. Intravascular target sites contemplated by the present invention are described in further detail below.

As used herein, the term “subject” can refer to any warm-blooded organism including, but not limited to, human beings, pigs, rats, mice, dogs, goats, sheep, horses, monkeys, apes, rabbits, cattle, etc.

As used herein, the term “treating” refers to therapeutically regulating, preventing, improving, alleviating the symptoms of, and/or reducing the effects of a cardiac disorder or disease, such as acute heart failure.

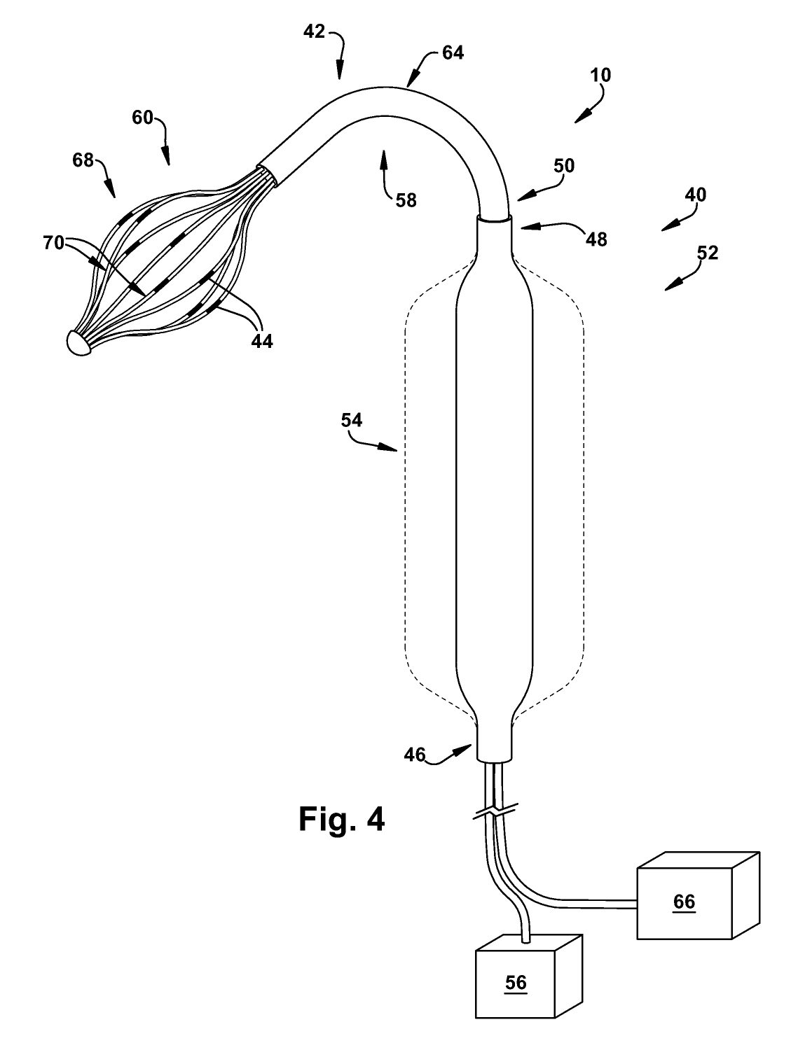

The present invention relates generally to devices and methods for modulating cardiac function, and more particularly to combined counterpulsation and neuromodulatory devices and methods that provide counterpulsation therapy with little or no need for inotropic pharmacologic support. As representative of one aspect of the present invention, FIGS. 4-7 illustrate a therapy delivery device 10 and related method for modulating cardiac function in a subject. The present invention is based, at least in part, on the discovery that electrical stimulation of mixed autonomic nerve targets can result in a positive inotropic effect of increased contractility with either no change or a decrease in heart rate. As explained in more detail below, the present invention advantageously provides therapy delivery devices, systems, and related methods that allow for the combined use of counterpulsation devices (e.g., intra-aortic balloon pumps or IABPs) and cardiac autonomic nerve modulation to simultaneously control the balance of sympathetic and parasympathetic autonomic control of heart rate and contractility, thereby decreasing or eliminating the need for inotropic pharmacologic support during IABP therapy for patients with heart failure.

A brief discussion of the neurophysiology is provided to assist the reader with understanding the present invention. The nervous system is divided into the somatic nervous system and the ANS. In general, the somatic nervous system controls organs under voluntary control (e.g., skeletal muscles) and the ANS controls individual organ function and homeostasis. For the most part, the ANS is not subject to voluntary control. The ANS is also commonly referred to as the visceral or automatic system.

The ANS can be viewed as a “real-time” regulator of physiological functions which extracts features from the environment and, based on that information, allocates an organism's internal resources to perform physiological functions for the benefit of the organism, e.g., responds to environment conditions in a manner that is advantageous to the organism.

The ANS conveys sensory impulses to and from the central nervous system to various structures of the body such as organs and blood vessels, in addition to conveying sensory impulses through reflex arcs. For example, the ANS controls constriction and dilatation of blood vessels; heart rate; the force of contraction of the heart; contraction and relaxation of smooth muscle in various organs; lungs; stomach; colon; bladder; visual accommodation, secretions from exocrine and endocrine glands, etc. The ANS does this through a series of nerve fibers, and more specifically through efferent (outgoing) and afferent (incoming) nerves. The ANS acts through a balance of its two components: the SNS and the PNS, which are two anatomically and functionally distinct systems. Both of these systems include myelinated preganglionic fibers which make synaptic connections with unmyelinated postganglionic fibers, and it is these fibers which then innervate the effector structure. These synapses usually occur in clusters called ganglia. Most organs are innervated by fibers from both divisions of the ANS, and the influence is usually opposing (e.g., the vagus nerve slows the heart, while the sympathetic nerves increase its rate and contractility), although it may be parallel (e.g., as in the case of the salivary glands). Each of these is briefly reviewed below.

The PNS is the part of the ANS controlling a variety of autonomic functions including, but not limited to, involuntary contraction or relaxation of muscles in the blood vessels, gut, bladder, rectum and genital organs, as well as glandular secretions from the eye and salivary glands. The vagus nerve is part of the PNS. Parasympathetic nerve fibers are contained within the last five cranial nerves and the last three spinal nerves and terminate at parasympathetic ganglia near or in the organ they supply. The actions of the PNS are broadly antagonistic to those of the SNS; lowering blood pressure, slowing heartbeat, stimulating the process of digestion etc. The chief neurotransmitter in the PNS is acetylcholine. Neurons of the parasympathetic nervous system emerge from the brainstem as part of the Cranial nerves III, VII, IX and X (vagus nerve) and also from the sacral region of the spinal cord via Sacral nerves. Because of these origins, the PNS is often referred to as the “craniosacral outflow”.

In the PNS, both pre- and post-ganglionic neurons are cholinergic (i.e., they utilize the neurotransmitter acetylcholine). Unlike adrenaline and noradrenaline, which the body takes around 90 minutes to metabolize, acetylcholine is rapidly broken down after release by the enzyme cholinesterase. As a result the effects of the PNS are quicker acting and quicker to reverse in comparison to the SNS.

Each pre-ganglionic parasympathetic neuron synapses with just a few post-ganglionic neurons, which are located near, or in, the effector organ, a muscle or gland. As noted above, the primary neurotransmitter in the PNS is acetylcholine such that acetylcholine is the neurotransmitter at all the pre- and many of the post-ganglionic neurons of the PNS. Some of the post-ganglionic neurons, however, release nitric oxide as their neurotransmitter.

The SNS is the part of the ANS comprising nerve fibers that leave the spinal cord in the thoracic and lumbar regions and supply viscera and blood vessels by way of a chain of sympathetic ganglia running on each side of the spinal column which communicate with the central nervous system via a branch to a corresponding spinal nerve. The SNS controls a variety of autonomic functions including, but not limited to, control of movement and secretions from viscera and monitoring their physiological state, stimulation of the sympathetic system inducing, e.g., the contraction of gut sphincters, heart muscle and the muscle of artery walls, and the relaxation of gut smooth muscle and the circular muscles of the iris. The chief neurotransmitter in the SNS is adrenaline, which is liberated in the heart, visceral muscle, glands and internal vessels, with acetylcholine acting as a neurotransmitter at ganglionic synapses and at sympathetic terminals in skin and skeletal muscles. The actions of the SNS tend to be antagonistic to those of the PNS.

The neurotransmitter released by the post-ganglionic neurons is nonadrenaline (also called norepinephrine). The action of noradrenaline on a particular structure, such as a gland or muscle, is excitatory in some cases and inhibitory in others. At excitatory terminals, ATP may be released along with noradrenaline. Activation of the SNS may be characterized as general because a single pre-ganglionic neuron usually synapses with many post-ganglionic neurons, and the release of adrenaline from the adrenal medulla into the blood ensures that all the cells of the body will be exposed to sympathetic stimulation even if no post-ganglionic neurons reach them directly.

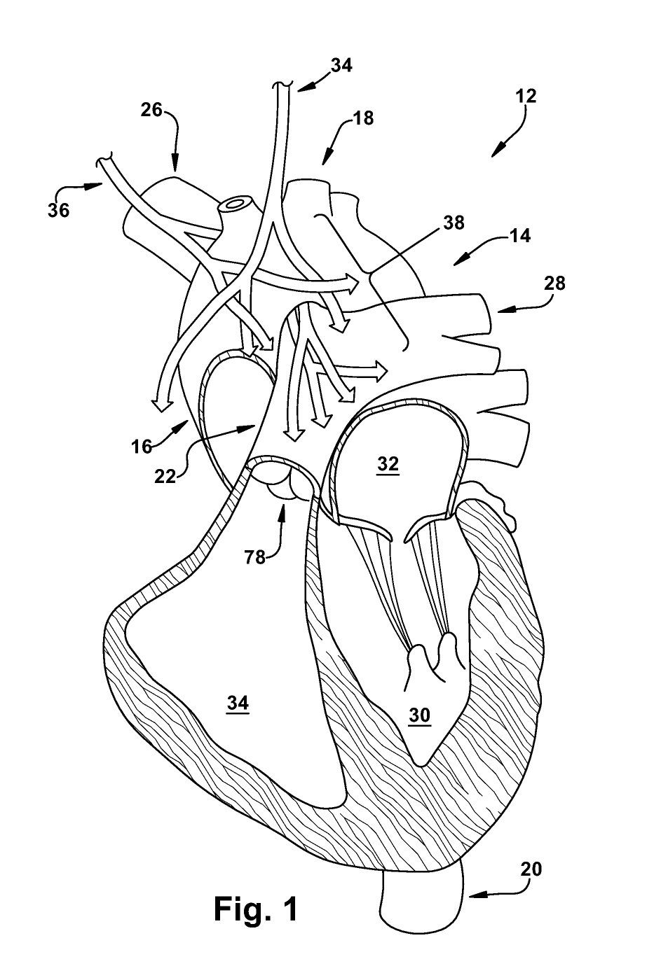

FIG. 1 is a schematic illustration of the human heart 12, the pulmonary artery 14, the ascending aorta 16, the aortic arch 18, and a portion of the descending aorta 20. As is known in the art, the pulmonary artery 14 includes the pulmonary trunk 22, which begins at the base of the right ventricle 24, the right pulmonary artery 26, and the left pulmonary artery 28. The pulmonary trunk 22 is short and wide, having a length of approximately 5 cm (2 inches) and a diameter of approximately 3 cm (1.2 inches). The pulmonary trunk 22 then branches into the left and right pulmonary arteries 28 and 26, which deliver deoxygenated blood to the corresponding lung (not shown).

The aorta (not shown in entirety) is the main trunk of a series of vessels that convey oxygenated blood to the body. The aorta commences at the upper part of the left ventricle 30, where it is about 3 cm in diameter and, after ascending for a short distance, arches backward and to the left side over the root of the left lung. The aorta then descends within the thorax (not shown) on the left side of the vertebral column (not shown), passes into the abdominal cavity (not shown) through the aortic hiatus (not shown) in the diaphragm, and ends opposite the lower border of the fourth lumbar vertebra by dividing into the right and left common iliac arteries (not shown).

The ascending aorta 16 is covered at its commencement by the pulmonary trunk 22 and the right auricular (not shown) and, higher up, is separated from the sternum (not shown) by the pericardium (not shown), the right pleura (not shown), the anterior margin of the right lung, some loose areolar tissue (not shown), and the remains of the thymus (not shown). Posteriorly, the ascending aorta 16 rests upon the left atrium 32 and the right pulmonary artery 26.

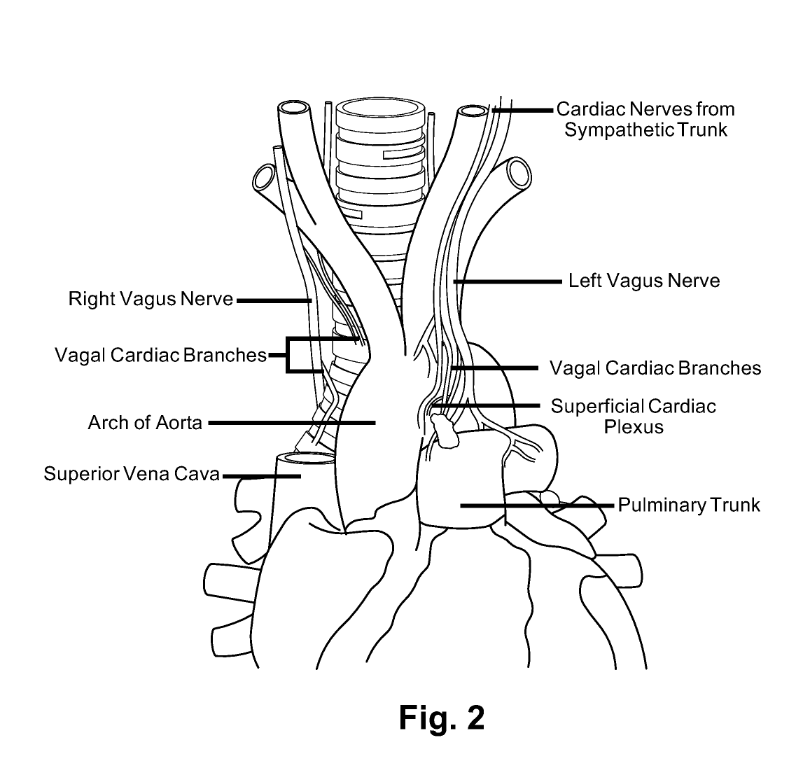

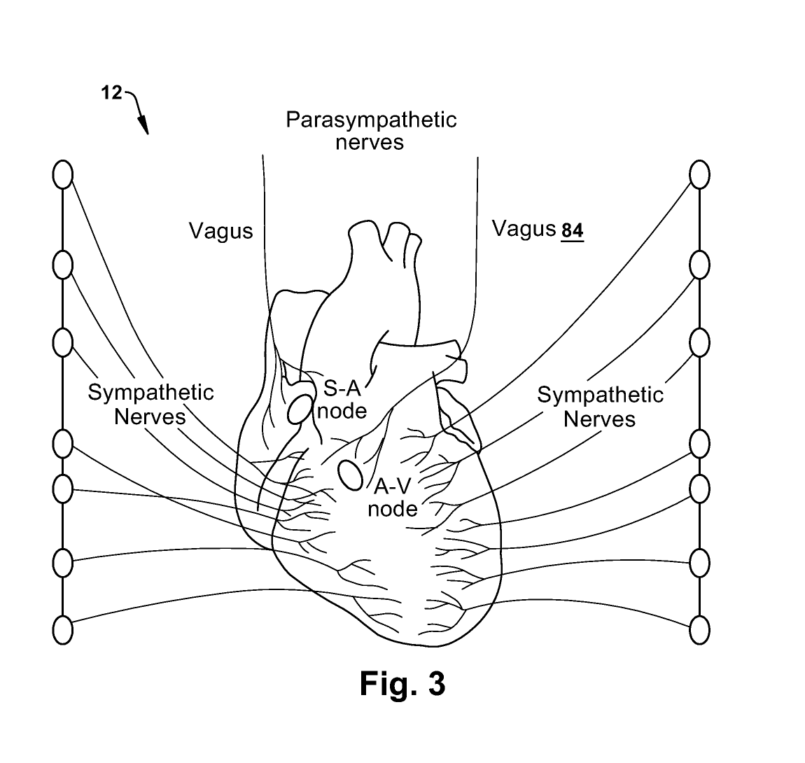

The aortic arch 18 is covered anteriorly by the pleura (not shown) and anterior margins of the lungs, and by the remains of the thymus. Passing downward on the left side of this part of the aortic arch 18 are four nerves: the left phrenic (not shown); the lower of the superior cardiac branches 34 of the left vagus 84 (FIGS. 2-3); the superior cardiac branch 36 of the right sympathetic (posterior to the aortic arch); and the trunk of the left vagus. As the last nerve crosses the aortic arch 18, it gives off its recurrent branch, which hooks around below the vessel and then passes upward on its right side. The highest left intercostal vein (not shown) runs obliquely upward and forward on the left side of the aortic arch 18, between the phrenic nerve (not shown) and the vagus nerve 84.

On the right are the deep part of the cardiac plexus 38, the esophagus (not shown), and the thoracic duct (not shown). The cardiac plexus 38 lies chiefly on the epivascular surface of the great vessels, in the concavity of the aortic arch 18 in front of the right pulmonary artery 26, in front of the bifurcation of the trachea above the point of division of the pulmonary artery 14, and behind the aortic arch. Above the aortic arch 18 are the innominate (not shown), left common carotid (not shown), and left subclavian arteries (not shown), which arise from the convexity of the aortic arch and are crossed close to their origins by the left innominate vein. Below the aortic arch 18 are the bifurcation of the pulmonary artery 14, the left bronchus (not shown), the ligamentum arteriosum (not shown), the superficial part of the cardiac plexus 38, and the left recurrent nerve (not shown).

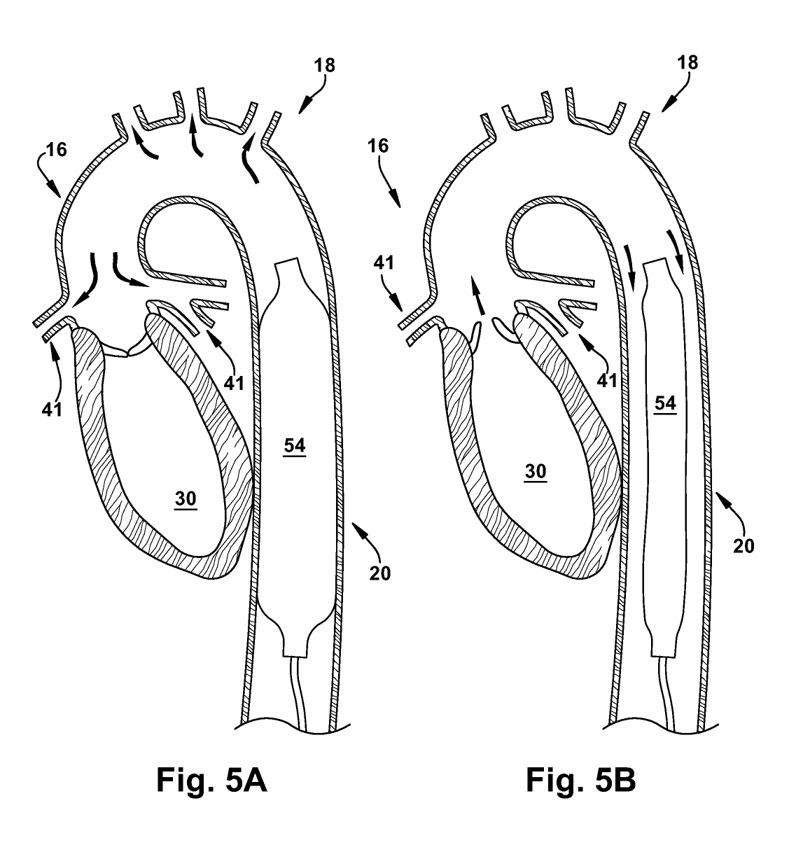

Referring to FIG. 4, one aspect of the present invention includes a therapy delivery device 10 for modulating cardiac function in a subject. By “modulating cardiac function”, it is meant that a particular cardiac function, such as heart rate, contractility, stroke volume, ejection fraction, cardiac output, ventricular workload (preload and afterload), etc., can be increased, decreased, or kept the same (e.g., stabilized) by the present invention. The therapy delivery device 10 includes a counterpulsation component 40, an endovascular lead 42, and at least one electrode 44 that is physically coupled with the endovascular lead. The counterpulsation component 40 can include any device or apparatus capable of decreasing cardiac afterload during cardiac systole and increasing pressure in the aorta and blood flow into the coronary arteries 41 (FIGS. 5A-B) during diastole. As shown in FIG. 4, the counterpulsation component 40 includes a proximal end 46 and a distal end 48, which is physically coupled to a proximal end 50 of the endovascular lead 42.

In one example of the present invention, the counterpulsation component 40 comprises an IABP 52. The IABP 52 comprises a mechanical device that increases coronary blood flow (diastolic augmentation) and decreases myocardial oxygen demand (systolic unloading). The IABP 52 includes a cylindrical balloon 54 (e.g., made of polyethylene) that is in fluid communication with an inflation source 56. When implanted, the IABP 52 sits in the aorta (FIGS. 5A-B), approximately 2 cm from the orifice of the left subclavian artery (not shown). Inflation and deflation of the balloon 54 are timed to counterpulse with the heart 12. As shown in FIGS. 5A-B, for example, the balloon 54 actively deflates in systole, which allows aortic systolic forward flow from the left ventricle 30 to effectively increase forward blood flow by reducing afterload. The balloon 54 then actively inflates in diastole, thereby pressurizing the space between the balloon and the closed aortic valve (not shown), which increases blood flow to the coronary arteries 41 (which originate from two of the aortic valve cusps). These actions combine to decrease myocardial oxygen demand and increase myocardial oxygen supply in the heart 12.

The endovascular lead 42 comprises a lead body 58 having a distal end 60, a proximal end 50, and a main body portion 64 extending between the distal and proximal ends. The proximal end 50 and a portion of the main body portion 64 comprise an outer, tubular sheath or housing made of a flexible, insulating, biocompatible and biostable material, such as silicone rubber or polyurethane. The proximal end 50 of the endovascular lead 42 can be further operably connected to an electrical stimulator 66 that is capable of delivering electrical energy (e.g., an electric current) to at least one electrode 44.

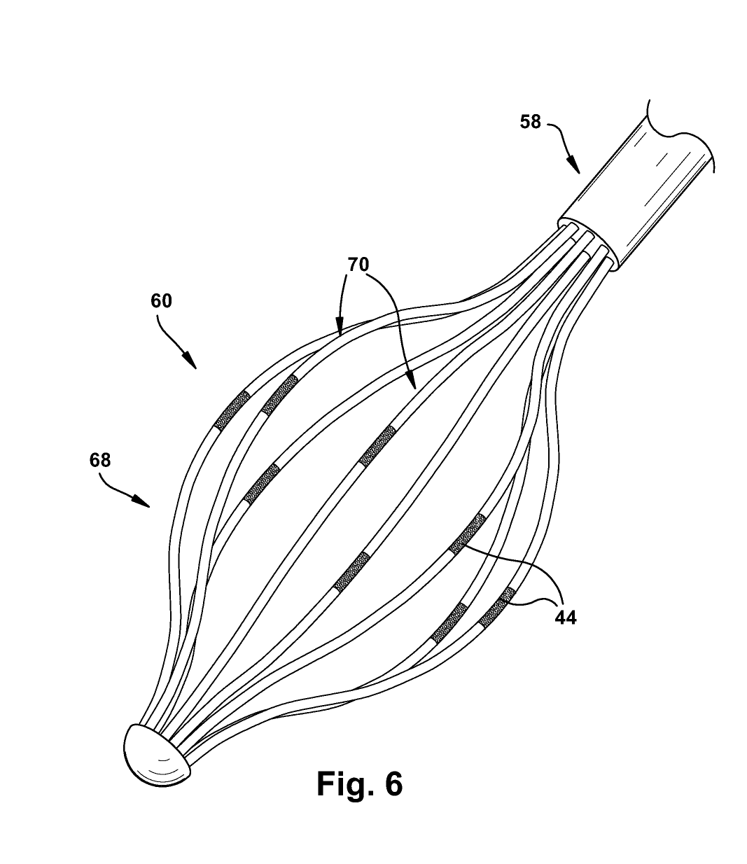

As shown in FIG. 4, the distal end 60 of the endovascular lead 42 includes an expandable frame 68 that can provide intimate contact between the endovascular lead and a vessel wall. The frame 68 is collapsible for fitting within a lumen of a delivery catheter (not shown) during insertion of the therapy delivery device 10 into the vasculature of a subject. Specifically, the frame 68 has a first collapsed configuration (not shown) that is smaller than the diameter of delivery catheter lumen and, when deployed, a second radially expanded configuration (FIG. 6) designed to contact the vessel wall against which the frame is positioned. The frame 68 can be made from a super-elastic material, such as Nitinol, which allows the frame to return to its expanded state when deployed from the delivery catheter and assume a collapsed state when retracted back into the delivery catheter.

The endovascular lead 42 includes at least one electrode 44 physically coupled thereto. Depending upon the particular configuration of the endovascular lead 42, the electrode(s) 44 can have any desired shape and size. For example, the electrode(s) 44 may have a triangular shape, a rectangular shape, an ovoid shape, and/or a band-like shape (e.g., a split band configuration). As described in more detail below, the electrode(s) 44 can be configured to have a unipolar or bipolar configuration. A unipolar configuration, for example, can include a configuration whereby current flows from a stimulating electrode (or electrodes) at a desired intraluminal or extravascular target site to a reference ground with a larger surface area distant from the target site (e.g., the body of a lead). Alternatively, in a bipolar configuration, the stimulating current flows between closely-spaced anode and cathode electrodes (e.g., lying on the same lead or catheter). For example, two or more electrodes 44 can be disposed on one or more of each of the electrical conductors 70 comprising the lead 42 to form a bipolar configuration.

One or more electrodes 44 can be arranged about the endovascular lead 42 to facilitate focal delivery of electrical energy to a mixed autonomic nerve target. Additionally or optionally, the entire surface area of the electrode(s) 44 may be conductive or, alternatively, only a portion of the surface area of the electrode(s) may be conductive. By modifying the conductivity of the electrode(s) 44, the surface area of the electrode(s) may be selectively modified to facilitate focal delivery of electrical energy to the mixed autonomic nerve target. The electrode(s) 44 may be made of any material capable of conducting electrical energy, such as platinum, platinum-iridium, or the like.

As shown in FIG. 6, the frame 68 includes a plurality of insulated electrical conductors 70, each of which is connected to a cylindrical electrode 44 disposed annularly thereon. There can be any number of conductors 70 having any number of electrodes 44 disposed thereon. The endovascular lead 42 has a unipolar configuration in FIG. 6; that is, one or more electrodes serve as stimulating electrodes while the lead body 58 serves as the ground. It will be appreciated, however, that other unipolar, bipolar, or multi-polar configurations are possible. It will also be appreciated that the electrodes 44 can be mounted at any position on the conductors 70.

Although the endovascular lead 42 illustrated in FIG. 4 and FIGS. 6-7 includes a frame 68, it should be appreciated that the endovascular lead can have any other configuration suitable for delivering electrical energy to a mixed autonomic nerve target. For example, the endovascular lead 42 can be configured in a similar or identical manner as known nerve stimulation leads, such as wire-like stimulation leads commercially available from Medtronic, Inc. (Minneapolis, Minn.), St. Jude Medical (St. Paul, Minn.), and Greatbatch Medical (Clarence, N.Y.).

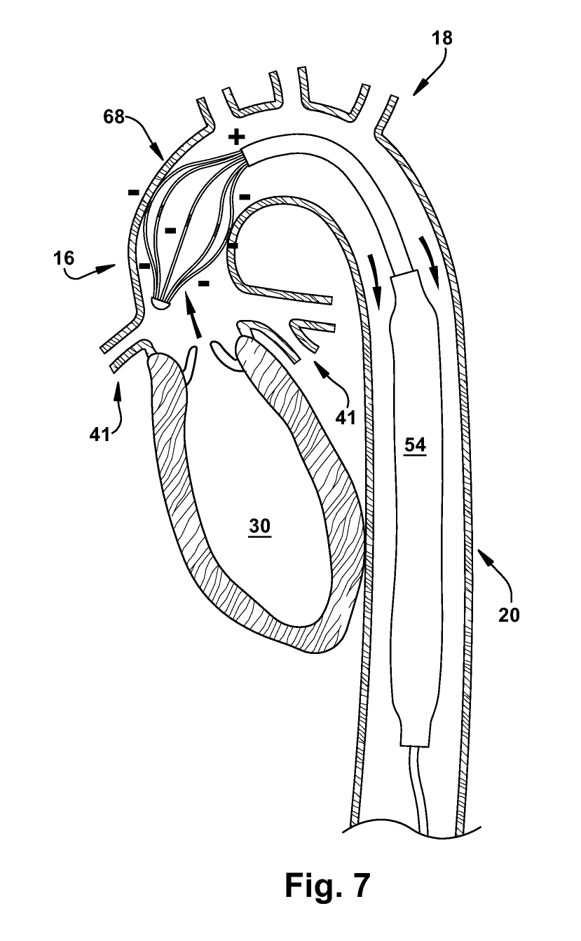

Another aspect of the present invention is illustrated in FIG. 7 and includes a method for modulating cardiac function in a subject. Although the method is described below in terms of treating a subject suffering from acute decompensated heart failure (ADHF), it will be appreciated that the method can be used to treat subjects suffering from a variety of other cardiac disorders or diseases. ADHF is most commonly due to left ventricular systolic or diastolic dysfunction, with or without additional cardiac pathology, such as coronary artery disease or valve abnormalities. ADHF is the primary diagnosis for more than one million hospital admissions annually in the U.S., and the secondary diagnosis for nearly 2 million hospitalizations. In 2003, the estimated direct and indirect costs for the treatment of heart failure totaled $29.6 billion. This situation indicates the current heart failure therapies fall short of achieving success.

To treat a subject suffering from ADHF, one step of the method includes providing a therapy delivery device 10. The therapy delivery device 10 can be identically or similarly constructed as the one illustrated in FIGS. 4-6. For example, the therapy delivery device 10 (FIG. 7) can comprise a counterpulsation component 40, such as an IABP 52 that is physically coupled to an endovascular lead 42. The distal end 60 of the endovascular lead 42 can include an expandable frame 68, as shown in FIG. 6. Further, the endovascular lead 42 (FIG. 7) can have a unipolar construction comprising a stimulating electrode and a reference ground (e.g., the lead body 58). Although not shown in FIG. 7, the therapy delivery device 10 can also be physically connected to an electrical stimulator 66 (FIG. 4) for delivering electrical energy to the electrodes 44 (FIG. 7), as well as an inflation source 56 (e.g., a pump) for delivering an inflation fluid (e.g., air or saline) to the balloon 54.

Next, the therapy delivery device 10 is inserted into the vasculature of the subject using, for example, a percutaneous or endovascular approach. Prior to inserting the therapy delivery device 10, however, the dimensions of the ascending aorta 16, aortic arch 18, and a superior portion of the descending aorta 20 can be determined to correctly size the therapy delivery device for optimal placement. Various methods and devices for determining the dimensions of the aorta are known in the art and include, for example, computed tomography, magnetic resonance imaging, angiography and fluoroscopy. After determining the dimensions of the aorta, an appropriately-sized therapy delivery device 10 is chosen. The therapy delivery device 10 is suitably sized, for example, so that the dimensions of the balloon 54 (i.e., in the expanded configuration) correspond to the dimensions of the superior portion of the descending aorta 20. Additionally, the endovascular lead 42 is appropriately-sized so that the dimensions of the frame 68 facilitate intimate contact with an intraluminal target site, such as the wall of the ascending aorta 16.

Percutaneous placement of the therapy delivery device 10 starts by accessing a blood vessel with a delivery device. For instance, a guidewire (not shown) may be introduced into the vasculature of the subject via a vascular opening or incision (not shown). Vascular access may be through a peripheral arterial access site (not shown), such as a left or right femoral artery. The guidewire is inserted through the incision into the left femoral artery, for example, in an antegrade direction. The guidewire can then be advanced into or near the left ventricle 30.

Next, the therapy delivery device 10 is placed in a delivery catheter (not shown) in a collapsed configuration and securely attached to a proximal end (not shown) of the guidewire. The delivery catheter is then advanced over the guidewire until the delivery catheter is suitably positioned in the aorta. In particular, the delivery catheter is positioned so that the balloon 54 of the IABP 52 is located in the superior portion of the descending aorta 20, just inferior to the aortic arch 18. Additionally, the delivery catheter is positioned so that the distal end 60 of the endovascular lead 42 is located in the ascending aorta 16 close to the site where the aorta arches over and contacts the pulmonary artery 14. As discussed above, this is the site where a portion of the cardiac plexus 38 traverses epivascular portions of both the ascending aorta 16 and pulmonary artery 14.

Once the therapy delivery device 10 is appropriately positioned in the vasculature of the subject, the delivery catheter is removed. Removal of the delivery catheter allows the frame 68 of the endovascular lead 42 to self-expand into intimate contact with the wall of the ascending aorta 16 to secure the endovascular lead therein. Further withdrawal of the delivery catheter also exposes the balloon 54, which is in the collapsed configuration. If it has not been done so already, the guidewire can be removed from the vasculature of the subject.

After the therapy delivery device 10 is positioned in the vasculature of the subject as shown in FIG. 7, operation of the IABP 52 can be commenced. Operation of the IABP 52 is commenced using an inflation source 56 (FIG. 4), such as a pump capable of selectively expanding and collapsing the balloon 54 (e.g., using a medium, such as air, helium or sterile saline) (FIG. 7). Inflation and deflation of the balloon 54 are timed to counterpulse with the heart 12. As discussed above, the balloon 54 actively deflates during systole, which allows aortic systolic forward flow from the left ventricle 30 to effectively increase forward blood flow by reducing afterload. The balloon 54 then actively inflates during diastole, thereby pressurizing the space between the balloon and the closed aortic valve to increase blood flow into the coronary arteries 41. These actions combine to decrease myocardial oxygen demand and increase myocardial oxygen supply in the heart 12 of the subject.

Either prior to, contemporaneous with, or following activation of the IABP 52, electrical energy is delivered to the electrodes 44 of the endovascular lead 42. Electrical energy can be delivered to the electrodes 44 continuously, periodically, episodically, or a combination thereof. Depending upon the configuration of the endovascular lead 42, electrical energy can be delivered in a unipolar, bipolar, and/or multipolar sequence or, alternatively, via a sequential wave, charge-balanced biphasic square wave, sine wave, or any combination thereof. Electrical energy can be delivered to all the electrodes 44 at once or, alternatively, to only a select number of desired electrodes. The particular voltage, current, and frequency delivered to the electrodes 44 may be varied as needed. For example, electrical energy can be delivered to the electrodes 44 at a constant voltage (e.g., at about 0.1 μV to about 50 V), at a constant current (e.g., at about 25 microampes to about 50 milliamps), at a constant frequency (e.g., at about 2 Hz to about 2,500 Hz), and at a constant pulse-width (e.g., at about 50 μsec to about 5,000 μsec).

As electrical energy is delivered to the endovascular lead 42, the electrodes 44 conduct electrical energy to the aortic wall at the intravascular target site, thereby modulating the activity of the various cardiac nerves that innervate the aortic wall and form a portion of the cardiac plexus 38. Where stimulating energy is delivered to the electrodes 44, for example, the cardiac nerves innervating the aortic wall are caused to fire action potentials, which can then propagate to the myocardium. Consequently, delivery of electrical energy at the intravascular target site (as shown in FIG. 7) results in a simultaneous increases coronary blood flow during ventricular diastole, a decrease in ventricular workload during systole, and an increase in myocardial contractility without increasing the heart rate of the subject. This is the net effect of simultaneous stimulation of sympathetic cardiac nerves, which increases contractility and heart rate, and parasympathetic cardiac nerves, which reduces heart rate.

It is known that the heart rate of ADHF patients is already high due to acute deterioration of cardiac function, and also that the heart rate of such patients is frequently increased by treatment with large doses of inotropic drugs. Additionally, it is known that high heart rate interferes with proper IABP function due to the dependence of IABP therapy on synchronization with the cardiac cycle. While it is easy to increase heart rates by drugs and/or pacemakers, it is very difficult to decrease heart rates. Advantageously, the present invention, which provides the beneficial hemodynamic effects of increasing contractility without a clinically significant increase in afterload and either no change or a decrease in heart rate, provides enhanced efficacy of IABP therapy.

Consequently, the present invention allows: ADHF patients to more quickly recover from a low cardiac output state (i.e., by improving IABP cardiac output) and improving myocardial perfusion and increasing myocardial contractility (i.e., by improving native ventricular cardiac output); a quicker transition to surgical/interventional modalities since patient outcomes are expected to improve with better hemodynamic pre-procedural status; and a decreased dosage or even elimination of inotropic drugs during IABP therapy, which aggravates myocardial ischemia and decreases or eliminates the negative side effects of these drugs (e.g., arrhythmias, hypotension, increased heart rate, and increased myocardial oxygen demand).

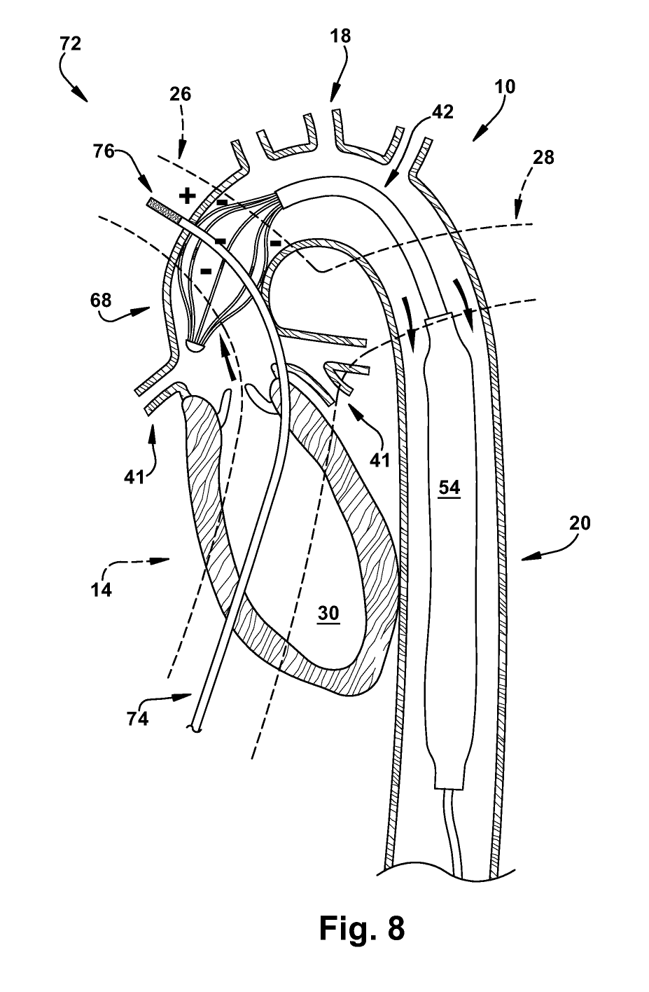

Another aspect of the present invention is illustrated in FIG. 8 and includes a system 72 and related method for modulating cardiac function in a subject. The system 72 includes a therapy delivery device 10 and a second endovascular lead 74. The therapy delivery device 10 can be identically or similarly constructed as the therapy delivery device described above. For example, the therapy delivery device 10 can include a counterpulsation component 40 (e.g., an IABP 52), a first endovascular lead 42, and a first electrode 44 that is physically coupled to the first endovascular lead. The second endovascular lead 74 can be similarly or identically constructed as the first endovascular lead 42. As shown in FIG. 8, for example, the second endovascular lead 74 can have a wire-like configuration and include a second electrode 76 that is physically coupled thereto. The second electrode 76 can have a polarity that is different than the polarity of the first electrode 44 upon application of electrical energy thereto. It will be appreciated that the second endovascular lead 74 can alternatively comprise a Swan-Ganz catheter (not shown) modified to incorporate an anode on the distal end thereof so that an additional catheter is not needed.

The system 72 can be used to treat a subject suffering from ADHF, for example. To do so, the therapy delivery device 10 can first be inserted into the vasculature of the subject (as described above). For example, the therapy delivery device 10 can be inserted into a peripheral arterial access site, such as the left femoral artery and then advanced through the vasculature until the first electrode 44 of the first endovascular lead 42 is in electrical communication with a mixed autonomic nerve target (e.g., a portion of the cardiac plexus 38), and the balloon 54 of the IABP 52 is located in a superior portion of the descending aorta 20 (i.e., inferior to the aortic arch 18). Operation of the IABP 52 can then be commenced as described above.

Either prior to, during, or after placement and operation of the therapy delivery device 10, the second endovascular lead 74 can be introduced into the vasculature of the subject using a guidewire (not shown), which can be inserted via a vascular opening (not shown). Vascular access may be through a peripheral venous access site (not shown), such as a femoral vein (not shown). The guidewire can then be inserted through the incision into the inferior vena cava (not shown) and advanced into the right atrium (not shown), across the tricuspid valve (not shown) into the right ventricle 24, and through the pulmonary valve 78 into the pulmonary artery 14.

Next, the second endovascular lead 74 is positioned in the pulmonary artery 14 so that the second electrode 76 is adjacent the first electrode 44. As shown in FIG. 8, the second endovascular lead 74 is positioned in the right pulmonary artery 26 so that the second electrode 76 is adjacent the first electrode(s) 44 comprising the first endovascular lead 42. After the second endovascular lead 74 is appropriately positioned, electrical energy can be delivered to each of the first and second electrodes 44 and 76 so that electrical energy is conducted to walls of the ascending aorta 16 and the pulmonary artery 14, respectively. As described above, activation of the therapy delivery device 10 results in a simultaneous increase in coronary blood flow during ventricular diastole, a decrease in ventricular workload during systole, and an increase in myocardial contractility without increasing the heart rate of the subject (e.g., the heart rate can remain the same or decrease). Consequently, the aspect of the present invention illustrated in FIG. 8 and described above permits the use of IABP therapy with lower or without inotropic pharmacologic support.

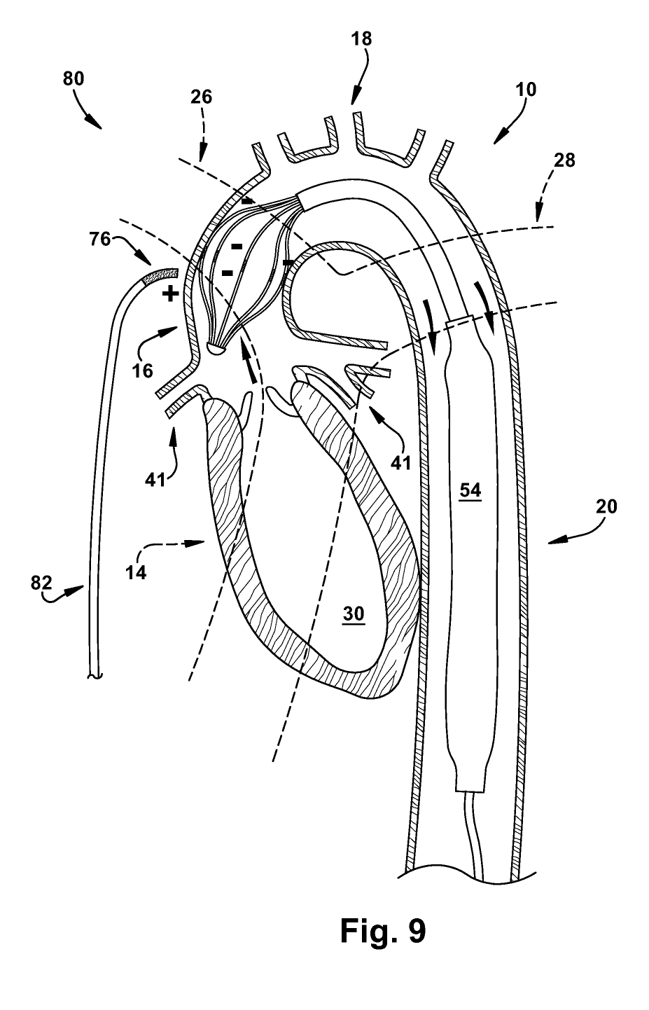

Another aspect of the present invention is illustrated in FIG. 9 and includes a system 80 and a related method for modulating cardiac function in a subject. The system includes a therapy delivery device 10 and an epivascular lead 82. The therapy delivery device 10 can be identically or similarly constructed as the therapy delivery device described above. For example, the therapy delivery device 10 can include a counterpulsation component 40 (e.g., an IABP 52), an endovascular lead 42, and a first electrode 44 that is physically coupled to the endovascular lead. The epivascular lead 82 can be similarly or identically constructed as the second endovascular lead 74 described above. As shown in FIG. 9, for example, the epivascular lead 82 can have a wire-like configuration and include a second electrode 76 that is physically coupled thereto. The second electrode 76 can have a polarity that is different than the polarity of the first electrode 44 when electrical energy is applied thereto.

The system 80 can be used to treat a subject suffering from ADHF, for example. To do so, the therapy delivery device 10 is first inserted into the vasculature of the subject (as described above). For example, the therapy delivery device 10 can be inserted into a peripheral arterial access site, such as the left femoral artery and then advanced through the vasculature until the first electrode 44 of the endovascular lead 42 is in electrical communication with a mixed autonomic nerve target (e.g., a portion of the cardiac plexus 38), and the balloon 54 of the IABP 52 is located in a superior portion of the descending aorta 20 (i.e., inferior to the aortic arch 18). Operation of the IABP 52 can then commenced as described above.

Either prior to, during, or after placement and operation of the therapy delivery device 10, the epivascular lead 82 can be surgically placed on or about an extravascular target site. The extravascular target site may be any suitable location inside the chest cavity for electrically modulating the sympathetic and parasympathetic autonomic cardiac fibers that innervate the heart 12. For example, the extravascular target site may be on the epivascular surface of the great vessels, such as between the pulmonary artery 14 and the aortic root or, more specifically, at the cardiac plexus 38. In some cases, the extravascular target site may be the pericardial transverse sinus (not shown in detail), which is a passage within the pericardial sac (not shown in detail) behind and between the aortic root and the pulmonary trunk 22, in front of the superior vena cava (not shown).

A surgical approach or procedure, such as an open-chest procedure, a mini-thoracotomy, or some other approach, such as a subxiphoid approach as disclosed in U.S. Pat. No. 7,247,134 to Vidlund et al., which is hereby incorporated by reference in its entirety, can be used to position the epivascular lead 82 at the extravascular target site. For example, the epivascular lead 82 can be positioned about the epivascular surface of the pulmonary artery 14 so that the second electrode 76 is in electrical communication with the mixed autonomic nerve target (e.g., a plurality of cardiac nerves forming the cardiac plexus 38). This will put the cardiac nerves running in the pulmonary artery 14 and/or aortic perivascular tissues directly in the stimulation path between the first and second electrodes 44 and 76.

After the endovascular and epivascular leads 42 and 82 are appropriately positioned, electrical energy can be delivered to each of the first and second electrodes 44 and 76 so that electrical energy is conducted to the aortic wall (i.e., in the case of the first electrode) and the cardiac nerves extending in the pulmonary artery 14 and/or aortic perivascular tissues (i.e., in the case of the second electrode). As described above, delivery of electrical energy to the mixed autonomic nerve target has the advantageous effect of simultaneously increasing coronary blood flow during ventricular diastole, decreasing ventricular workload during systole, and increasing myocardial contractility without increasing the heart rate of the subject (e.g., the heart rate can remain the same or decrease). Consequently, the aspect of the present invention illustrated in FIG. 9 and described above permits the use of IABP therapy with lower or without inotropic pharmacologic support.

It will be appreciated that certain aspects of the present invention illustrated in FIGS. 8-9 can be configured to improve electrical stimulation efficiency. One parameter affecting the efficiency of electrical stimulation includes the proximity between an anode and a cathode and, thus, the pairing of anode and cathode can be subject to significantly differing end effects if one of the pair is displaced. To mitigate or prevent displacement and improve electrical stimulation efficiency, a magnet (not shown) can be placed on or near each of the first electrode 44 and the second electrodes 76 of the epivascular lead 82 and the second endovascular lead to magnetically couple the electrodes during operation of the systems 72 and 80. Magnetic coupling between the first electrode 44 and the second electrodes 76 of the epivascular lead 82 and the second endovascular lead would ensure close proximity and provide a measure of resistance to displacement, thereby optimizing electrical stimulation efficiency.

It will also be appreciated that stimulation of extravascular tissues carrying a plurality of cardiac nerve fibers that are not visible to the eye for direct stimulation may require an array of electrodes (not shown) to determine the optimum anode(s) and cathode(s) to be used for producing the optimum desired cardiovascular effect. For extravascular tissues, such as the cardiac plexus 38 (which contains both sympathetic and parasympathetic nerve fibers and ganglia), an electrode array can be used to select the desired balance of sympathetic and parasympathetic cardiac stimulation (along with concomitant intravascular stimulation).

The following example is for the purpose of illustration only and is not intended to limit the scope of the claims, which are appended hereto.

### EXAMPLE

Experiments for extravascular stimulation of cardiac nerves were performed on twelve dogs. After opening the chests of the dogs under general anesthesia, a stimulating catheter electrode was placed epivascularly in the cardiac plexus near the right pulmonary artery behind the ascending aorta. An electrical signal was provided to the electrode with the stimulation frequency set at 20 Hz, the pulse width set at 4 msec, and the voltage ranging between 10 V and 50 V. Hemodynamic and epicardial echocardiographic data were recorded with and without stimulation using a left ventricular conductance catheter, Swan-Ganz catheter, and left atrial pressure line.

In all twelve dogs, the systolic aortic and systolic left ventricular pressures, its dP/dt, and left ventricular stroke work increased with stimulation (p<0.0001 for all the parameters) with cardiac output increasing from 2.9±1.0 to 3.4±1.0 L/min (p=0.001). The end-systolic elastance and preload recruitable stroke work, which are load-independent indices of left ventricular contractility, significantly increased with stimulation (from 1.2±0.4 to 1.5±0.5 mm Hg/ml, p=0.0001; and from 30.1±11.0 to 39.3±7.8 mm Hg, p=0.003, respectively), suggesting an increase in left ventricular contractility. Left ventricular ejection fraction with echocardiography significantly increased from 50.5±7.7 to 57.6±8.2% (p=0.012). Heart rate (from 101±20 to 97±21 beat/min), central venous pressure, pulmonary arterial pressure, and left atrial pressure remained unchanged (p>0.1). There was a slight but statistically significant increase in systemic vascular resistance (from 1,426±339 to 1,574±250 dyne·sec·m⁻⁵, p=0.04) and a decrease in pulmonary vascular resistance (from 161±64 to 127±9 dyne·sec·m⁻⁵, p=0.01). These results demonstrate that electrical neuromodulation can selectively increase ventricular contractility with minimal changes in heart rate, systemic vascular resistance, and/or pulmonary vascular resistance.

From the above description of the invention, those skilled in the art will perceive improvements, changes and modifications. Such improvements, changes and modifications within the skill of the art are intended to be covered by the appended claims.

Having described the invention, the following is claimed:

1. A therapy delivery device for modulating cardiac function in a subject, said therapy delivery device comprising:
a counterpulsation component having a proximal end and a distal end;
an endovascular lead having a proximal end and a distal end, said proximal end of said endovascular lead being physically coupled to said distal end of said counterpulsation component; and
at least one electrode physically coupled with said endovascular lead.

2. The therapy delivery device of claim 1, wherein said counterpulsation component is an intra-aortic balloon pump (IABP).

3. The therapy delivery device of claim 1, wherein said distal end of said endovascular lead comprises an expandable frame.

4. The therapy delivery device of claim 1, wherein said at least one electrode has a unipolar configuration comprising an active electrode and a reference electrode.

5. A system for modulating cardiac function in a subject, said system comprising:
a therapy delivery device comprising:
a counterpulsation component having a proximal end and distal end;
a first endovascular lead having a proximal end and a distal end, said proximal end of said first endovascular lead being physically coupled to said distal end of said counterpulsation component; and
a first electrode having a first polarity and being physically coupled with said first endovascular lead; and
a second lead having a second electrode physically coupled thereto, said second electrode having a second polarity that is different than the first polarity.

6. The system of claim 5, wherein said counterpulsation component is an IABP.

7. The system of claim 5, wherein said distal end of said first endovascular lead comprises an expandable frame.

8. The system of claim 5, wherein said at least one electrode has a unipolar configuration comprising an active electrode and a reference electrode.

9. The system of claim 5, wherein said second lead is an endovascular lead.

10. The system of claim 5, wherein said second lead is an epivascular lead.

11. A method for modulating cardiac function in a subject, said method comprising the steps of:
providing a therapy delivery device, the therapy delivery device comprising a counterpulsation component, an endovascular lead, and at least one electrode, the counterpulsation component having a proximal end and a distal end, the endovascular lead having a distal end and a proximal end, the proximal end of the endovascular lead being physically coupled to the distal end of the counterpulsation component, the at least one electrode being physically coupled to the endovascular lead;
positioning the therapy delivery device in the vasculature of the subject so that the at least one electrode is in electrical communication with a mixed autonomic nerve target and the counterpulsation component is located in a portion of the descending aorta; and
activating the therapy delivery device;
wherein activation of the therapy delivery device simultaneously increases coronary blood flow during ventricular diastole, decreases ventricular workload during systole, and increases myocardial contractility without increasing the heart rate of the subject.

12. The method of claim 11, wherein the subject is suffering from acute decompensated heart failure (ADHF).

13. The method of claim 11, wherein said step of positioning the therapy delivery device further comprises the step of placing the endovascular lead in a portion of the ascending aorta such that the at least one electrode is in electrical communication with the mixed autonomic nerve target.

14. The method of claim 11, wherein the heart rate of the subject remains the same upon activation of the therapy delivery device.

15. The method of claim 11, wherein activation of the therapy delivery device decreases the heart rate of the subject.

16. A method for modulating cardiac function in a subject, said method comprising the steps of:
providing a system comprising a therapy delivery device and a second endovascular lead, the therapy delivery device comprising a counterpulsation component, a first endovascular lead, and a first electrode, the counterpulsation component having a proximal end and a distal end, the first endovascular lead having a distal end and a proximal end, the proximal end of the first endovascular lead being physically coupled to the distal end of the counterpulsation component, the first electrode having a first polarity and being physically coupled to the first endovascular lead, the second endovascular lead being physically coupled to a second electrode and having a second polarity that is different than the first polarity;
positioning the therapy delivery device in the vasculature of the subject so that the first electrode is in electrical communication with a mixed autonomic nerve target and the counterpulsation component is located in a portion of the descending aorta;
positioning the second endovascular electrode in a blood vessel so that the second electrode is adjacent the first electrode; and
activating the system;
wherein activation of the system simultaneously increases coronary blood flow during ventricular diastole, decreases ventricular workload during systole, and increases myocardial contractility without increasing the heart rate of the subject.

17. The method of claim 16, wherein the subject is suffering from ADHF.

18. The method of claim 16, wherein said step of positioning the second endovascular lead further comprises positioning a distal end thereof in the pulmonary artery.

19. The method of claim 16, wherein said step of positioning the therapy delivery device further comprises the step of placing the first endovascular lead in a portion of the ascending aorta such that the first electrode is in electrical communication with the mixed autonomic nerve target.

20. The method of claim 16, wherein the heart rate of the subject remains the same upon activation of the therapy delivery device.

21. The method of claim 16, wherein activation of the therapy delivery device decreases the heart rate of the subject.

22. A method for modulating cardiac function in a subject, said method comprising the steps of:
providing a system comprising a therapy delivery device and an epivascular lead, the therapy delivery device comprising a counterpulsation component, an endovascular lead, and a first electrode, the counterpulsation component having a proximal end and a distal end, the endovascular lead having a distal end and a proximal end, the proximal end of the endovascular lead being physically coupled to the distal end of the counterpulsation component, the first electrode having a first polarity and being physically coupled to the endovascular lead, the epivascular lead including a second electrode that is physically coupled thereto and has a second polarity that is different than the first polarity;
positioning the therapy delivery device in the vasculature of the subject so that the first electrode is in electrical communication with a mixed autonomic nerve target and the counterpulsation component is located in a portion of the descending aorta;
percutaneously positioning the epivascular lead so that the second electrode is adjacent the first electrode; and
activating the system;
wherein activation of the system simultaneously increases coronary blood flow during ventricular diastole, decreases ventricular workload during systole, and increases myocardial contractility without increasing the heart rate of the subject.

23. The method of claim 22, wherein said step of positioning the therapy delivery device further comprises the step of placing the first endovascular lead in a portion of the ascending aorta such that the first electrode is in electrical communication with the mixed autonomic nerve target.

24. The method of claim 22, wherein the subject is suffering from ADHF.

25. The method of claim 22, wherein the heart rate of the subject remains the same upon activation of the therapy delivery device.

26. The method of claim 22, wherein activation of the therapy delivery device decreases the heart rate of the subject.
